(19) United States
(12) Patent Application Publication
(10) Pub. No.: US 2004/0072790 A1
(43) Pub. Date: Apr. 15, 2004

(54) Safe natural pharmaceutical composition for treating cancer

(76) Inventor: Yaguang Liu, Flushing, NY (US)

Correspondence Address: L. Y. Research Co., 67-08 168th Street, Flushing, NY, 11365, US

(21) Appl. No.: 10/267,084

(22) Filed: Oct. 9, 2002

(51) Int. Cl. A61K 31/739; A61K 31/55; A61K 31/366

(52) U.S. Cl. 514/54; 514/214.03; 514/460

(57) ABSTRACT

The present invention related to the natural drug of Homoharringtonine (HHT) combined with other ingredients which include Matrine (MAT), Apigenin (APN), Yejuhua lactone (YLE), Lipopolysaccharide of Kelp (LIK), Puerarin (PUN) and Indirubin (IND) for diverting human cancer cells to closely normal cells, inducing apoptosis of cancer cells and inhibiting cancer cells. Specifically, this invention provides a new method for producing HHT and other ingredients

### BACKGROUND OF THE INVENTION

The present invention related to the natural drug of Homoharringtonine (HHT) combined with other natural ingredients which include Matrine (MAT), Apigenin (APN), Yejuhua lactone (YLE), Lipopolysaccharide of Kelp (LIK), Puerarin (PUN) and Indirubin (fND) for diverting human cancer cells to closely normal cells, inducing apoptosis of cancer cells and inhibiting cancer cells growth Specifically, this invention provides a new method for producing HHT and relative ingredients.

### DESCRIPTION OF THE PRIOR ART

HHT was used with success in the U S and China in the treatment of acute and chronic leukemia (Susan O'Brien et al Blood, vol 93, No 12, 1999, pp4149-4153) HHT is extracted from skins, stems, leaves and roots of Cephalotaxus fortunei Hook and other related species, such as Cephalotaxus sinensis Li, C hainanensis, and C. wilsoniana. However, growth of these plants is slowly and concentration of HHT in these plants also is extremely low (typically 0 01%). Alternative sources are needed to meet the increasing demand. So far, the total chemical synthesis of HHT is not available for commerce and industry. For the reasons given above, cell culture and partial synthesis of HHT is an important new source. On the other hand, a great development in the oncology, enabled by progression of molecular and cellular technologies throughout the culminating in 1990s was transformed from killing both cancer and normal cells to induce diverting cancer cells to resemble normal cells and do not injure the normal cells

Cancer is the second leading cause of death in the United States, and the incidence of cancer continues to climb annually In recent years, about 1 million new cases of cancer are diagnosed yearly in the U.S About half million people and 7 million people of annual deaths are in the US and in the world, respectively A lot of anticancer drugs including chemical and antibiotics have effects to kill cancer cells. But it also kills off some normal human cells, appears many kinds of side effects, among them the inhibition of bone marrow and tract are the most common

Cyclopho-sphamide, for example, is a chemotherapeutic drug, which is highly effective against a wide range of human cancer Cyclophosphamide established a role in the treatment of some major cancer types including Lymphomas, Acute Lymphatic leukemia, Chronic Lymphatic Leukemia, Breast, Pulmonal, Ovarial cancer and Tumor or marrow mulciple, Osseous, Sarcoma etc Unfortunately, Cyclophosphamide has high toxicity, for example, it does damage to hemotopoietic organs, alimentary tract and decrease immune function. The toxicity of other anti-cancer medicines, for example, Fluorouracil, Mustine and 6-Mercaptopurine, etc is higher than Cyclophosphamide

Some antibiotics are effective anticancer drugs, for example, Adriamycin is used for treatment of some cancers including leukemia, gastric pancreatic, breast cancer, etc. A prime limit factor to the administration of Adriamycin is cardiotoxicity The most serious side effect of Adriamycin administration is myocardial degeneration causing congestive heart failure. This acute cardiomyopathy may cause acute left ventricular dysfunction, arrhythmia and myocardial infarction. Adriamycin induced cardiomyopathy is thought to be permanent and rapidly progressive Late cardiomyopathy develops in weeks, months or even years Some patients were reported to have developed progressive cardiomyopathy two and two-half years after receiving this drug

Obviously, to overcome toxicity of anti-cancer is very important. Many agents have been suggested to reduce or prevent toxicity of anticancer medicines. For example, Vitamin E and N-acetyl-L-Cysteine have also been reported to be effective in preventing Adriamycin cardiotoxicity Although more recent research showed evidence to contradict these findings, in that neither of vitamin E and N-acetyl-L-Cysteine prevents adriamycin-induced cardiotoxicity. p The major antiviral drugs can inhibit viral replication but also inhibit some host cell function and possess serious toxicity For example, Amantadine, Idoxuridine, Cytarabine, and Vidarabine are major antiviral drugs using in clinic now Amantadine can inhibit influenza A The most marked toxic effects of Amantadine are insomnia, slurred speech, dizziness, ataxia and other central nervous system sign Idoxuridine can inhibit the replication of herpes simplex virus in the cornea However, DNA synthesis of host cells is also inhibited. Cytarabine can inhibit DNA viruses But it also inhibits immune function in human. By weight it is about 10 times more effective than Idoxuridine, and it is also 10 times more toxic for host cell Vidarabine can inhibit herpes-virus, but it is also produces more marked adverse gastrointestinal or neurologic side effects.

Many reports indicated that the side effects of plant's anticancer drugs are lower than chemical and antibiotic's anticancer drugs Therefore, the development of plant drug has progressed very fast now. Taxol, for example, is a novel anticancer plant drug isolated from the needles and bark of the western yew, Taxus brevifolia It is the prototype for a new class of antitumor drugs, which are characterized by their capacity to promote the assembly of microtubules Clinical trials conducted in the late 1980s and early 1990s demonstrated impressive clinical activities against advanced ovarian and breast cancer. Taxol, however, still has some side effects, for example, myelosuppression, diarrhea, emesis, oligospermia, cellular depletion in lymphoid tissues, changes in serum hepatic enzymes, and elevations in cholesterol and triglycerides were observed. More important, taxol has two big problems. The first problem is that natural source of taxol is very limited And the second problem is that taxol is a poor-water soluble Vehicles for parental administration on taxol cause serious side effects

HHT is a new anticancer drug because it has lower side effects, and can divert human cancer cells to closely normal cells, induce apoptosis and inhibit growth of cancer cells. More important, if HHT combined with other ingredients, which include MAT, APN, YLE, or PI K, PUN and TND, it treats cancer more effectively and is more safely.

### DETAILED DESCRIPTION OF THE INVENTION

The present invention disclosed that HHT induced diverting of cancer cells, including leukemia cells and gastric and other cancer cells to resemble normal cells HHT also inhibits growth and induced apoptosis of cancer cells. Diverting cancer to normal cells and apoptosis is important goal of cancer therapy.

The present invention provides a new method for culture plant tissue. New culture method for production of HHT provides cells with high HUT productivity and special elicitation which caused by derivative of gibberellin. The new culture method significantly increased the content HHT In general, many useful products of natural ingredients by plant cell stored within the cells thus making their efficient product very difficult The amounts and rates of production of these useful products are very low. Our method induces secretion of HHT by cells into the culture. Therefore, present invention provided a new method for production HHT on large scale.

Phenylalanine and tyrosine are precursor for biosynthesis of HHT. This scheme predicted that HHT should be generated from two molecules of phenylalanine or tyrosine. Therefore, added phenylalanine and tyrosine into culture system is very important for increasing producing of HHT More important is elicitor for producing of HHT in culture. The present invention disclosed the gibberellin's derivative is effective elicitor. Semi-synthesis HHT is another new source HHT can be synthesis from large and inactive natural alkaloids

To add some effective drugs combined with HHT can significantly increase anticancer effects of HHT. These drugs include Matrine (MAT), apigenin (APN), Yejuhua lactone (YLE), Lipopolysaccharide of Kelp (LIK), Puerarin (PUN) and Indirubin (IND). The experimental data showed that above drugs have strong synergisms effects for treating leukemia and other cancer cells and more safe. These new drugs are synergists for HHT.

The following specific examples will provide detailed illustrations of methods of producing relative drugs, according to the present invention and pharmaceutical dosage units containing demonstrates its effectiveness in treatment of cancer cells These examples are not intended, however, to limit or restrict the scope of the invention in any way, and should not be construed as providing conditions, parameters, reagents, or starting materials which must be utilized exclusively in order to practice the present invention.

### EXAMPLE 1

Production of HHT by Cell Culture of Cephalotaxus

So far, HHT is extracted from stems and skins of Cephalotaxus species. However, growth of plant of Cephalotaxus species is very slow and concentration of HHT in plant is extremely low. Furthermore, it is difficult to harvest the plants because of their low propagation rate and the danger of drastic reduced in plant availability. Also, cost of total chemical synthesis of HHT is very expensive and is not available for commerce now For the reasons given above it is more difficult to obtain Cephalotaxus on a large scale for long time Therefore, Cephalotaxus cell cultures are one of best methods for obtaining HHT.

In this present invention, special elicitation is disclosed and it will significantly increase production of HHT

Method(s)

Cell and Tissue Culture

Parts of stems, leaves, skins or roots of Cephalotaxus species were surface disinfected by treatment in 70% ethanol for 10 minutes and followed by 0.1 HgCl₂ for 3 minutes. Plant materials were washed five times for 10 minutes each by sterilized water. Parts of plant were cut into small pieces (0 5-1 mm) and put pieces to Murashige and Skoog's (MS) medium and supplemented with derivative of gibberellin, phenylalamine (PHE), tyrosine (TYR) naphthaleneacetic acid (NAA), Kinetin (3 mg/L), and 3% sucrose (w/v). PH of medium was adjusted to 5 7˜5 8. Agar (10 g/L) added to medium. Callus tissues were collected from agar media and suspension cultured cells were harvested by filtration and cultured in MS medium. The cultures were kept in a culture room at 26° C.±1° C. Friable callus tissues were obtained. Approximately 100 of the callus was inoculated into 4 L of MS liquid medium containing sucrose, derivative of gibberellin, PUE, TYR, NAA and Kinetin Then callus tissues were cultivated 26° C. for 35 days on rotary shaker operated at 120 rpm in the dark. Cells were subcultured into fresh medium of same composition every 2 weeks and maintained at 120 rpm at 26°±1° C. Packed cell volume (PCV), fresh weight (FW), dry weight (DW), concentration of HHT and concentration of sugar were determined every 5t h day. The cells were harvested and dried

### EXAMPLE 2

HHT Extracted from Plant Tissue

Extraction of LIHT has several major methods which including extraction by organic solvent, chromatograph and adjust pH

HHT was extracted from plant tissue culture, plant cells or the skins, stems, leaves, seeds and roots of Cephalotaxus species 1 kg of ground Cephalotaxus fortunei Hook was extracted with 8 liters of 90% ethanol at room temperature for 24 hrs. Filtered the solution to yield a filtrate A and filtercake. Percolated the filtercake with ethanol and filter again to yield filtrate B. Combined A and B, and distilled under reduced pressure to recover ethanol and an aqueous residue. To this residue, added 2% HCl to adjust the pH to 2 5 Separated the solids from the solution by filtration to yield a filtrate C Washed the solids once with 2% HCl and filtered to yield a filtrate D Combined C and D and adjusted the pH to 9.5 by adding saturated sodium carbonate solution Extracted the alkaline filtrate with chloroform and separated the chloroform layer from the aqueous layer. Repeated this extraction process five times. Combined all the chloroform extracts and distilled at reduced pressure to recover chloroform and alkaloid as a solid residue respectively The solid alkaloid was then dissolved in 6% citric acid in water. The solution was divided into three equal portions These were adjusted to pH 7, 8 and 9 by adding saturated sodium carbonate solution. The portions having pH 8 and 9 were combined and extracted with chloroform The chloroform extracts were distilled under reduced pressure, whereby chloroform was removed and recovered and crude HHT was obtained. The crude HHT was dissolved in pure ethanol and crystallized The crystals were refined by recrystallization in diethyl ether.

The portion having a pH of 7 passed through a liquid chromatographic column packed with alumina of diameter to height 1 50 The column was finally flushed with chloroform and followed by chloroform-methanol of 9 1 mixture The resulting alkaloids were mixture of HHT. The mixed HHT was then separated from each other by countercurrent distribution employing chloroform and pH 5 buffer. The first fraction of the countercurrent distribution was HHT. HHT was purified by crystallization in methyl alcohol and dried under vacuum. HHT has the following chemical structure

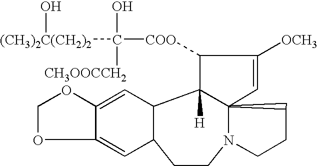

Yield 0 02%. Melting point: 144°-146° C. Infrared spectrum 3500, 3400, 1665, 1030 and 940 cm-. Ultraviolet spectrum λpeak alcohol mμ (logλ) 240 (3 55), 290 (3.61)

### EXAMPLE 3

Semi-Synthesis of HHT

HHT shows a significant inhibitory activity against leukemia and other cancer. Concentration of HHT, however, has only 0 01% in natural sources Cephalotazine (CEP) is major alkaloids present in plant extracts and the concentration of Cephalotaxus has about 1%. Therefore, concentration of CEP is about 100 times higher then HHT in plant sources. But CEP is inactive. Therefore, synthesis of HHT from CEP will increase large additional sources of HHT.

(1) Extraction of CEP

1 kg of dried stems, leaves or roots of Cephalotaxus species were milled, placed in a percolator, along 8 L of 95% of ethanol, and allowed to stand 24 hours The ethanol was recovered under reduced pressure (below 40° C.) 2 L of 5% tartaric acid was added to concentrated ethanol solution The ammonia water was added to the acidic solution and adjusted pH to 9. The solution of pH 9 was filtered and yielded a filtrate The filtrate was extracted with CHCl₃. CHCl₃ was recovered under reduced pressure and residue was obtained. The residue was chromatographed packed with alumna and eluted by CHCl₃—MeOH (9:1). Eluate was concentrated under reduced pressure. Residue was dried under vacuum. The product is CEP.

(2) Synthesized HHT from CEP

General

Melting points were determined on a Fisher-Johns apparatus Infrared spectra were obtained on a Perkin-Elmer 567 infrared spectrophotometer or on a Beckman 4230 IR spectrophotometer. Peak positions were given in cm⁻¹ The IR spectra of solid samples were measured as potassium bromide dispersions, and the spectra of liquids were determined in chloroform or carbon tetrachloride solutions. NMR spectra were measured on a Varian A-60, Perkin-Elmer R-32, Varian EM-390, or Bruker WH-90 NMR spectrometer Chemical-shift values were given in parts per million downfield from Me₄Si as an internal standard. Mass spectra were run on an AEI MS-12 Finnigan 3300, or CEC21-110B mass spectrometer Preparative thin-layer chromatography was accomplished using 750-μm layers of aluminum oxide HF-254 (type E), aluminum oxide 60 PF-254 (type E), silica gel HF-254 (type 60 PF-254), or silica gel GF-254 Visualization was by short-wave ultraviolet light. Grace silica gel, Grade 923, and Woelm neutral aluminum oxide, activity III, were used for column chromatography. Analytical thin-layer chromatography was run on plastic sheets precoated with aluminum oxide F-254 neutral (type T), 200-μm thick, and on Polygram Sil G/UV254 (silica gel), 250 μm on plastic sheets. Visualization was usually by short-wave ultraviolet light, phosphomolybdic acid, or iodoplatinate

α-ketoester-Harringtonine

10 mg of Benzene-α-acetone Na was put into 100 ml of benzene. Mixture was stirred at room temperature then was dissolved in 100 ml of pyridine and stirred at 0° C. Oxalic chloride was added from a dropping funnel to solution of pyridine. Stirring was continued while the solution warmed to room temperature and stand overnight Excess reagent was removed. This solution was dissolved in CH₂Cl₂ and cooled to near 0° C. in an ice water bath. 50 mg of CEP, 25 ml of CH₂Cl₂ and 25 ml of pyridine were added to cold CH₂Cl₂ solution. Manipulations were done in a dry N₂ atmosphere and all glassware heat-dried just before use. The suspension was stirred at room temperature and overnight The mixture (2) was washed with 10% Na₂CO₃ and saturated aqueous NaCl, then dried with auhydrous magenesium sulfate, and filtered and the solvents were removed in vacuo Evaporation provided as an amorphous solid α-ketoester-harringtonine (mp 143˜145° C.).

Semi-Synthesis of HHT

100 ml of CH₃CHBrCOOEt and activated zin dust and THF were added to the α-ketoester-harringtonine (at −78° C.) for 6 hours followed by slow warming to room temperature with stirred The reaction mixture was diluted with 100 ml CHCl₃ and 100 ml H₂O and solid Na₂CO₃ was added. CHCl₃ was evaporated under reduced pressure and residue was obtained

The residue was purified by chromatography on alumina The column was finally flushed with chloroform and followed by chloroform-methanol (9 1). The solvents were recovered under reduced pressure to provide as a solid Solid was dissolved in pure ethanol and crystallized. The crystals were refined by recrystalization in diethyl ether The crystals dried under vacuum The product is HHT

[α]_{D}−119° (C=0 96), MSm/e (%): 689 (M⁺, 3), 314 (3), 299 (20), 298 (100), 282 (3), 266 (4), 20 (3), 150 (8), 131 (12), 73 (18)

### EXAMPLE 4

Extraction of Matrine (MAT)

MAT was extracted from root of Sophora flavescens Ait 1 kg of ground plant was extracted 5 liters of methanol for 12 h at room temperature. The resulting methanol extract was filtered Methanol was then recovered under reduced pressure distillation. A distillated residue was dissolved in 300 ml of HCl and adjusted the PH to 3.5. NaOH was added to HCl solution and adjust pH to 13. Solution of pH 13 was extracted by CH₂Cl₂ and then CH₂Cl₂ was recovered under reduced pressure distillation The residue was dissolved in CHCl₃ Diethyl ether was added to CHCl₃. The mixture was filtered. The filtrate was concentrated to syrup under reduced pressure distillation. The residue passed through a chromatographic column packed with alumina again. The column was eluted with oil ether-acetone. The elution was concentrated under reduce pressure The residue was passed through a chromatographic column packed with alumina again. The column was eluted with benzol-oil ether. The organic solvent was recovered under reduced pressure and residue obtained. The acetone was added to residue and crystallized The crystals were refined by recrystallization in acetone The crystals were dried under vacuum and were found to have a meting point of 76° C. and [α]_{D}+39.1° (H₂O).

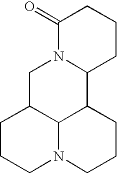

### EXAMPLE 5

Extraction of Apiin (APN)

Apiin was extracted from stems, leaves of Apium graveolens L. 1 kg of ground of plant was extracted with 5 liters in cold ethanol for 12 h. The resulting ethanol extracted was filtered Filtrate was concentrated under reduced pressure distillation. Mixture (1) was filtered Filtrate was cold and precipitate was formed. The mixture (2) was filtered. The filtercake was washed with diethyl ether The mixture (3) was filtered. The filtercake was washed with acetone The mixture (4) was filtered The filtercake was dissolved in hot water Pb (OAC)₂ was added to hot water. The mixture (5) was filtered, NaOH was added to the filtrate and precipitate was formed The mixture (6) was filtered. The filtercake was washed by water and was suspended on EtOH. H₂S was gassed to suspension of EtOH The mixture (7) was filtrated. The filtrate concentrated under reduced pressure and crystals were formed Crystals were refined by recrystallization in cold ethanol The crystals was dried under vacuum and were found to have melting point of 230˜232° C. and [α]_{D}−130° C. (C=0 12 CH₃OH)

Melting point 345˜350° C. UVλ^{EtOH} nm (ε) 269 (18800), 300 (13500), 340 (20900) MSm/e. 270 (100), 269 (s), 242 (s), 241 (m), 153 (s), 152 (s), 133 (w), 128 (w), 129 (w), 124 (s), 123 (s), 121 (s), 118 (s) ¹³CNMR (DMSO-d₆, 66 63 MH₂, TMS). 164 1 (c-2), 102.8 (c-3), 181.8 (c-4), 157.3 (c-5), 98 8 (c-6), 163 7 (c-7), 94 0 (c-8), 161 5 (c-9), 103.7 (c-10), 121 3 (c-1′), 128.4 (c-2′), 116 1 (c-3′), 161 1 (c-4′), 116.0 (c-5′), 128 4 (c-6′).

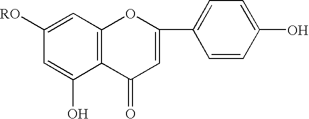

### EXAMPLE 6

Extraction of Indirubin (IND)

Indirubin was extracted from stems and leaves of Baphicanthus cusia (Ness) Bremek, or Isatis tinctoria L, or Isatis indigotica Fort, and Polygonum tinctorium Ait.

Isolation of Isatin B

10 kg of dried powder of plant was extracted with hot water 20 liters The extract was filtered The filtercake extracted with methanol (10 liter) Methanol was recovered under reduced pressure and residue obtained. The residue was extracted with chloroform (5 liter). The chloroform was recovered and the chloroform residue was chromatographed on silica gel G (1 kg), using chloroform as developing solvent The eluate was concentrated and rechromatographed on silica gel G (500 g) with chloroform as solvent. The IND was crystallized from chloroform and recrystallized and then dried under vacuum

Melting point 356˜358° C. UVλ^{CHCl3} nm 242, 292, 362, 540 IRλ^{KBr} cm⁻¹ 3345, 1670, 1620, 750

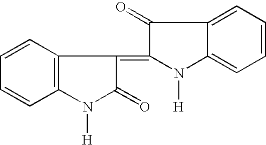

### EXAMPLE 7

Extraction of Yejuhua-Flavonoid (YLE)

Plants of Chrysanthemum indicum I, were dried and powdered 1 kg of the powder was dipped in 2 liters of 95% ethanol for about 24 hours at room temperature. The solution of ethanol was filtered and the extract filtrate was saved 2 liters of 95% ethanol was added to residue and refluxed in water bath and refluxing for 6 hours. The refluxed ethanol was cooled and filtered and the filtrate combined with the extract filtrate Ethanol was then recovered by reduced pressure distillation to remove the ethanol Residue was saved. 1,000 ml of acetic ether was added to residue and refluxed in a water bath for 6 hours. The refluxing procedure was repeated Acetic ether was then concentrated under reduced pressure distillation Crystals were formed Crystals were washed with water Final crystals were dried under vacuum and were found to have a melting point of about 250° C.

### EXAMPLE 8

Extraction of Lipopolysaccharide of Kelp (LIK)

2,000 ml of water was added to 1,000 g of dry powder of Kelp. The mixture was heated to boil and simmered for one and one-half hours after boiling This water extraction was repeated once and the two extracts were combined and filtered. The filtrate was concentrated under reduced pressure to approximately 500 ml and 95% ethanol was added to the concentrate to yield a final alcohol concentration of 60% The resulting solution was filtered to recover a precipitate which was dissolved in an appropriate amount of water, the resulting solution was filtered to remove residue and a filtrate was saved The filtrate was concentrated under reduced pressure to 200 ml and 95% ethanol added to the concentrate to yield a final alcohol concentration of 80% The solution was then discarded and the precipitate was washed three times with 95% ethanol and then twice with acetone and ether consecutively. The powder was dried under vacuum, and the resulting product was polysaccharides of Kelp.

### EXAMPLE 9.

Extraction of Puerarin (PUN)

Puerarin was extracted from Pueraria lobata (willd.) Ohwi or Pueraria thomsanii Benth. The roots of Pueraria lobata (willd.) Ohwi or Pueraria thomsanii Benth dried and powdered. One kilogram (1 kg) of powder was dipped in 5 liters 95% ethanol. The extraction was repeated twice with fresh 95% ethanol Extract combined Extract was concentrated to syrup under reduced pressure and ethanol was recovered Residue dried at 70° C. Drying-syrup was chromatographed on alumina using water-saturated butanol as the developing solvent to separate into ten color bands which was seen under ultraviolet light (U.V ) Then using butanol-Pyridine as the developing solvent systems From the fifth band (from the bottom) developing solvent was concentrated under reduced pressure and colorless crystals (m.p. 187° C.) were obtained Crystals were recrystallized from 50% ethanol. The final product was Pureatin and derivatives thereof

### EXAMPLE 10

Effect of HHT and in other Drugs on Differentiation of Human Leukemic Cells

Methods

Cell Lines

HL-60 cells were established from a patient with acute myeloid leukemia. The cells were cultured in culture flasks with RPMI plus 10% FCS

Studies of Induction of Differentiation

Differentiation of HL-60 cells was assessed by their abilities to produced superoxide as measured by reduction of NBT, by NSE staining and by morphology as detected on cytospin preparations stained with Diff-Quick stain Set, and by analysis of membrane-bound differentiation markers with two-color immunofluorescence Briefly, cells were preincubated at 4° C. for 60 min in 10% human AB serum and then with FITC-conjugated mouse IgGI isotype control Analysis of fluorescence was performed on a flow cytometer.

Clonogenic Assay in Soft Aga

HL-60 cells were culture in a two-layer soft agar system for 10 days without adding any growth factors as described previously, and colonies were counted using an inverted microscope The analo(,ues were added to the agar upper layer on day 0 For analysis of the reversibility of inhibition of proliferation, the cells were cultured in suspension culture with and without HHT or other drugs. After 60 hours, the culture flasks were gently jarred to loosen adherent cells, the cells were washed twice in cultured medium containing 10% FCS to remove the test drugs, and then the clonogenic assay was performed. NBT % indicated percentage of normal cells

These results were periodically confirmed by fluorescence microscopy and by DNA fragmentation

TABLE 1
Effect of drugs on cellular diversion of human leukemic cells

| Group | NBT (%) |
|---|---|
| N | 98 ± 12 |
| C | 5.0 ± 0.6 |
| HHT | 65 ± 7.0* |
| HHT + IND | 82 ± 9.8* |
| HHT + MAT | 79 ± 9.5* |
| HHT + YLE | 75 ± 8.7* |
| HHT + LIK | 72 ± 8.0* |
| HHT + APN | 69 ± 8.3* |
| HHT + PYE | 77 ± 9.5* |

N: Normal cells; C Human leukemic cells
* P < 0.01 compared with control group
Concentration of IND, MAT, YLE, LIK, APN and PYE is 50 ng/ml.
NBT (%) is index of normal cells The higher NBT (%) means higher normal cells.

Data of Table 1 showed that HHT could significantly induce diversion of leukemic cells to normal cells. And other drugs increasing induce diversion of leukemic cells to normal cells by HHT.

Results of other methods are similar data of Table 1.

### EXAMPLE 11.

Effect of HHT on Cellular Diversion of Gastric Cancer Cells

The gastric cancer cells and normal cells were cultured in PRMI 1640 medium supplement with 10% FCS serum Other method is similar to example 10

TABLE 2
Effect of HHT on diversion of gastric cancer cells

| Group | NBT (%) |
|---|---|
| N | 95 ± 14 |
| C | 8 ± 1.2 |
| HHT | 58 ± 8.9* |
| HHT + IND | 72 ± 8.0* |
| HHT + MAT | 65 ± 7.5* |
| HHT + YLE | 70 ± 9.2* |
| HHT + LIK | 64 ± 8.2* |
| HHT + APN | 60 ± 8.0* |
| HHT + PYE | 73 ± 9.5* |

*P < 0.01 compared with control group

Data of Table 2 showed that HHT could significantly induce diversion of gastric cancer cells to normal cells And other drugs increasing induce differentiation of gastric cancer cells by HHT

### EXAMPLE 12.

Effect of HHT and other Drugs on Apoptosis of Cancer Cells Methods

Human leukemia cells (HL-60) were grown in RPMI Medium 1640 supplemented with 10% (v/v) heat-inactivated FBS (56° C. for 30 min) at 37° C. in a humidified 95% air/5% CO₂ atmosphere. Cells were seeded at a level of 2×10⁵ cells/ml Cells were allowed to attain a maximum density of 1 2×10⁶ cells/ml before being passed by dilution into fresh medium to a concentration of 2×10⁵ cells/ml Cell pellets containing 5×10⁶ cells were fixed with 2 5% glutaraldehyde in cacodylate buffer (pH 7 4), dehydrated through graded alcohol, and infiltrated with LX-112 epoxy resin. After overnight polymerization at 60° C. 1-μm sections were cut with glass knives using a microtome. The sections were stained with 1% toluidine blue and coverslipped In addition, experimental examples were stained with May-Grunwald-giemsa stain for the demonstration of apoptosis

Determination of Apoptosis

Method (I) Apoptosis was Quantitated by Flow Cytometry

Cells (2.5×1060 were incubated in 10 ml IMDM plus 105 heat-inactivated fetal calf serum. Samples were incubated for 24 hours with various concentrations of drugs Control samples received the same amount of media, without drug addition After 24 hours of incubation the samples were pelleted and fixed in ethanol 705 for 15 minutes at 4° C., after three washes in PBS, the cells were treated with PNase I 0.5 mg/ml for 15 minutes at 37° C. The cells were harvested by centrifugation and resuspended in 50 μg/ml propidium iodide in PBS. Analysis (upon acquisition of 10,000-20,000 events) was performed on a FACscan flow cytometer with the FL2 detector in logarithmic mode, using Lysis II software (Becton Dickinson). Apoptotic cells were located in the hypodiploid region of the histogram, due to chromosome condensation and fragmentation

For evaluation of apoptosis by flow cytometry, cells were fixed and permeabilized in 1% paraformaldehyde and ice-cold 70% ethanol Digoxigenin-dUTP was incorporated at the 3′OH ends of the fragmented DNA in the presence of terminal deoxynucleotidyltranserase, and the cells were incubated with FITC-labeled anti-digoxigenin-dUTP and with propidium iodide. Green (apoptotic cells) and orange (total DNA) fluorescence were measured with a FACScan flow cytometer and analyzed with LYSIS It and CELLFIT programs Data were analyzed by Student's t-test P values were considered significant when <0.05.

Method (2) DNA Electrophoresis

Untreated and treated HL-60 cells collected by centrifugation, washed in phosphate buffered saline and re-suspended at a concentration of 5×10⁶ cells and 0.1% RNase A. The mixture was incubated at 37° C. for 30 min and then incubated for an additional 30 min at 37° C. Buffer was added and 25 μl of the tube content transferred to the Horizontal 1.5% agarose gel electrophoresis was performed at 2 V/cm DNA in gels visualized under UV light after staining with ethidium Bromide (5 μg/ml)

DNA fragmentation assays DNA cleavage was performed, quantitation of fractional solubilized DNA by diphenylamine assay and the percentage of cells harboring fragmented DNA determined by in labeling techniques For the diphenylamine assay, 5×10⁶ cells were lysed in 0 5 mL lysis buffer (5 mmol/L Tris-HCI, 20 mmol/L DTA, and 0 5% Triton X-100, pH 8 0) at 4° C. Lysates were centrifuged (15,000 g) for separation of high molecular weight DNA (pellet) and DNA cleavage products (supernatant). DNA was precipitated with 0.5 N perchloric acid and quantitated using diphenylamine reagent. The cell cycle distribution was determined 4 hours after addition of drug and represents mean±SD of 5 independent experiments

Method (3)

Apoptosis of HL-60 cells was assessed by changes in cell morphology and by measurement of DNA nicks using the Apop Tag Kkt (Oncor, Gaithersburg, MD). Morphologically, HL-60 cells undergoing apoptosis possess many prominent features, such as intensely staining, highly condensed, and/or fragmented nuclear chromatin, a general decrease in overall cell size, and cellular fragmentation into apoptotic bodies These features make apoptotic cells relatively easy to distinguish from necrotic cells. These changes are detected on cytospin preparations stained with Diff-Quick Stain Set Apoptotic cells were enumerated in a total of about 300 cells by light microscopy

TABLE 3
Effect of drugs on apoptosis of cancer cells (1)

Apoptosis (%)

| | Normal cells | Human leukemia cells |
|---|---|---|
| No drug | 8.1 ± 2.0 | 4.0 ± 0.9 |
| HHT (10 ng/ml) | 16.5 ± 2.0* | 33.8 ± 4.8** |
| HHT (50 ng/ml) | 18.5 ± 2.5* | 42.8 ± 5.2** |
| HHT (100 ng/ml) | 22.0 ± 3.5* | 60.2 ± 8.9** |
| HHT (500 ng/ml) | 29.5 ± 5.2* | 92.0 ± 10.8** |

*P < 0.01 compared with group of normal cells
**P < 0.01 compared with group of human leukemia cells

TABLE 4
Effect of drugs on apoptosis of cancer cells (2)

Apoptosis (%)

| | Normal cells | Human leukemia cell |
|---|---|---|
| HHT (50 ng/ml) | 18.5 ± 2.5 | 42.8 ± 5.2 |
| HHT + LIK | 16 ± 2.5 | 71 ± 8.0* |
| HHT + MAT | 15 ± 2.4 | 60 ± 8.2* |
| HHT + YLE | 12 ± 2.5 | 53 ± 7.9* |
| HHT + APN | 10 ± 2.0 | 49 ± 6.8** |
| HHT + IND | 9 ± 1.8 | 52 ± 5.9** |
| HHT + PYE | 12 ± 3.0 | 51 ± 6.0** |

Concentration of LIK, MAT, YLE, APN, UND and PYE is 50 ng/ml.
*P < 0.01 compared xvith group of human leukemic cells
**P < 0.05 compared with group of human leukemic cells

Data showed that HHT could significantly induce apoptosis of cancer cells. The effects of various drugs combination with fHHT (50 ng/ml) on normal and human leukemia cells are showed in the Table 3-4 Other drugs exerted a synergistic effect with HHT in increasing apoptosis of human leukemia cells and decreasing apoptosis of normal cells (Table 4). It means that LIK, MAT, YLE, APN, UND and PYE exerted an additive effect with HHT in treating human leukemia and protective normal cells

### EXAMPLE 13.

Effects of Drug on Tumor Cells Proliferation

Materials and Methods

Human tumor cell lines Hela leukemia HL-60, malignant melanocarcinoma B16, oral epidermoid carcinoma (KB), lung carcinoma (A549), breast carcinoma MCF-7, adenocarcinoma of stomach Animal tumor cell lines Walker carcinoma, LLC-WRC-256, malignant melanoma (RMMI 1846), 3T3, and S-180 sarcoma (CCRF-180). All lines were routinely cultured in the RPMI1640 medium supplemented 20% fetal calf serum. The experiment was carried out in 96 microplate, each well had 5×10⁵ cells and given desired concentration of 1 μg /ml (1×10⁻⁶ g/ml) drug Then the plate was incubated at 37° C. in an atmosphere of humidified air enriched with 5 percent carbon dioxide for 72 hours

Inhibition percent rate of tumor cell proliferation was obtained according to the bellow formula

$$Inhibition percent rate = \frac{Control - Test}{Control} \times 100 \%$$

Results

TABLE 5
Effect of drugs on inhibiting growth cancer cells

| Group | Inhibition (%) |
|---|---|
| No drug | — |
| HHT | 73.8 ± 8.1 |
| HHT + IND | 89.9 ± 12.5 |
| HHT + YLE | 79 ± 15.4 |
| HHT + MAT | 85 ± 16.8 |
| HHT + LIK | 84 ± 16.3 |
| HHT + APN | 75 ± 12.9 |
| HHT + PYE | 78 ± 19.8 |

Data of Table 5 showed that HHT could significantly inhibit human cancer cells proliferation and other drugs could increase effect of inhibiting cancer cells by HHT.

### EXAMPLE 14.

The Effect of HHT and other Drugs on the Growth of Transplanted Tumor

Experimental Procedure

Male mice, weight 20-22 g, were used in the experiment 1×10⁷ tumor cells were injected to mouse and other drugs injected intraperitoneally began second day. All mice were sacrificed on the 12th day, isolated the tumor and weighed and calculated the inhibition rate of tumor weight

Results

The effect of other drugs and HHT on the growth of animal transplanted tumor as illustrated by the Table 6 20 mg/kg of other drugs could inhibit the growth of L615 transplanted tumor.

TABLE 6
Effect of drugs on inhibition of transplanted tumor

| Group | Inhibition (%) |
|---|---|
| No drug | — |
| HHT | 68 ± 7.2 |
| HHT + IND | 89.8 ± 12.0 |
| HHT + YLE | 72.8 ± 9.5 |
| HHT + LIK | 70.5 ± 8.9 |
| HHT + MAT | 82.7 ± 10.5 |
| HHT + APN | 73.2 ± 9.8 |
| HHT + PYP | 73.8 ± 11.5 |

Data of Table 6 showed that HHT could significantly inhibit animal transplanted tumor and other drugs could increase effect of inhibiting cancer cells by HHT.

### Example 15.

The Effect of Drug on Decreasing of Tyrosine Kinase

The development of cancer cells can be viewed as a defect in the normal process of differentiation and disorder balance between proliferation and maturation that occurs in normal cells The expression of oncogenes plans a very important role in regulate cellular proliferation The tyrosine kinase (TK) is a protein product of expression of oncogenes. The TK catalyze the transfer of phasphate from ATP to the hydroxyl residues on protein substrates Activity of the TK is essential for the malignant transformation of cells. Hunter and Sefton demonstrated that the protein product of the src oncogene was a TK. In subsequent years, a number of oncogenes have been found to code for TK Such as src, yes, fgr, abl, erbB, mos, neu,fms, fps, ros and sis are considered to act through tyrosine kinase activity TK activity is strongly correlated with the ability of retroviruses to transform cells Also, maturation with reduced TK activity has lower transforming efficiency. Transformation of the HL-60 leukemia cells causes the high TK activity In fact, TK activity is enhanced in many human cancers, such as breast carcinomas, prostate cancer cells, colon cancers, and skin tumor The results of a lot of experiments indicated that tyrosine phosphorylation is an important intracellular mediator of proliferation and differentiation. Mature of cells possess relatively low levels of TK activity Similar TK activity is also related with the cellular receptors for several growth factors such as EGF, platelet-derived growth factor, insulin, and growth factor

In general, very low levels of TK are expressed in normal cells and high levels of TK are expressed in cancer cells Many evidences have been accumulated that the dysfunction of cellular oncogenes is a cause of human cancers Therefore, a drug, which inhibits the activity of TK, can provide a new way to overcome cancer. In other words, the development of effective inhibitors of TK can be used for the treatment of cancer.

Materials and Methods

[³²P] ATP and other isotopes were purchased form Amersham Corp. All other chemicals were reagent grade obtained from commercial suppliers

Cells: L1210 and P388 cells were grown at 37° C. on medium RPMI-1640 without antibiotics and supplemented with 10% horse serum. Cultures were diluted daily to 1×10⁵ cells/ml with fresh growth medium From a culture initiated with cells from ascitic fluid obtained from a mouse 5 days after implantation with in vivo-passage leukemia, a stock of ampoule containing 107 cells/ml in growth medium plus 10% dimethyl sulfoxide was frozen and stored in liquid nitrogen Cultures were started from the frozen stock and were passage for no more than 1 month

L1210 and P388 cells were grown at 37° C. on medium RPMI-1640 supplemented with 10% calf serum, 10,000 unit/ml of Penicillin and 10,000 unit/ml of Streptomycin. 1×10⁶/ml cells were placed in culture with different concentrations of HHT. Then the cell suspension was incubated at 37° C. in a humidified atmosphere of 5% CO₂-95% air for the indicated time. Reactions were terminated by addition of 3 ml of cold Earle's buffer. Cells were lysed, precipitated with 10% trichloroacetic acid (TCA) and filtered onto glass fiber filters. The filters were washed with phosphate-buffered saline and placed in scintillation vials, and radioactive emissions were counted.

Tyrosine kinase (TK) Assay. TK was measured by a modification of the method of Braun et al Briefly, H-60 leukemia cells were plated at a density of 5×10⁵ cells in 60-nm dished, and divided control and treatments groups for incubation 24 hours at 37° C. with 5% CO₂. The cells were collected by scraping, washed twice with phosphate-buffered saline, and resuspended at density of 10⁶ cells/ml in 5 mM HEPESs buffer (pH 7.4). The cells were then resuspended in 1 ml of buffer containing 5 mM HEPES (pH 7.6), 1 mM MgCl₂ and ImM EDTA, then placed on ice bath The cell membrane was disrupted by ultra sound and centrifuged at 1,000×g for 10 minutes The supernatant was ultra centrifuged at 30,000×g for 30 minutes at 4° C. The pellet was resuspended in 0 3 ml of buffer containing 25 mM HEOES, centrifuged at 12,000×g for 5 minutes The resulting supernatant was used for TK assay. Content of protein was determined 10 μg of protein placed in 20 mM HEPES (pH 7 6), 15 mM MgCl₂, 10 nM ZnCl₂ and 5% (v/v) nonidet P-40, with or without substrate [glutamic acid (GT), mg/ml]. After 5 minutes incubation at 25° C., the reaction was initiated by the addition of 25 μM [γ³²P] ATP (3 ci/mmol) After 10 minutes, the reaction was stopped by the addition of 20 mM cold ATP. 50 μl of the mixtures were spotted on glass microfiber filter discs and washed three times with cold trichloroacetic acid (TCA), contained 1 mM sodium pyrophosphate Air dried. Radioactivity was determined by liquid scintillation spectrometry. The net TK activity was determined after correcting for endogenous TK activity

Results and Discussion

The present study clearly demonstrated that HHT reduction in TK activity. A concentration-dependent inhibition was seen HHT caused a relatively strong inhibition, with inhibition 99.9% occurring at a concentration of 10⁻⁶ M

TABLE 7
Effect of HHT on TK activity of HL-60 leukemia cells

| Drugs | Concentration (M) | % of control activity |
|---|---|---|
| No drug | — | 100 |
| HHT (1) | 10⁻⁶ | 0.6 |
| HHT (2) | 10⁻⁷ | 21.5 ± 4.0 |
| HHT (3) | 10⁻⁸ | 87.8 ± 12 |

TABLE 8
Effects of drugs on TK activity

| Group | % of control activity |
|---|---|
| No drug | 100 |
| HHT | 21.5 ± 4.0 |
| HHT + IND | 40.0 ± 6.5** |
| HHT + YLE | 23.5 ± 4.5* |
| HHT + LIK | 26.5 ± 4.3* |
| HHT + MAT | 31.0 ± 5.2** |
| HHT + APN | 29.8 ± 3.5* |
| HHT + PYE | 32.8 ± 3.8** |

*P < 0.01 compared with HHT
**P < 0.05 compared with HHT

Data represent the mean of three experiments each done in duplicate, the range was less than 5% of the mean

HHT inhibited the HL-60 TK activity by 0 6% at 10⁻⁶ M, 78 5% at 10⁻⁷ M and 12.2% at 10⁻⁸ M

Table 7-8 indicated, HHT can significantly inhibit TK activity and other drugs increase the effect of decreasing TK activity of HHT.

### EXAMPLE 16.

Drug Inhibited Tumor Incidence In Vivo

The capacity of tobacco-specific nitrosamine 4-(methylnitrosamino)-1-(3-pyridyl)-1-butanone (NNK) to induce tumor incidence was recognized several years ago.

Methods

Every group had 20 mice For treatment group, each mouse was gave Drug by injection at dose of 20 mg/kg daily. For control group, each mouse was gave same volume of physiological saline Three days later, mice were gave 10 μmol NNK (in 0.1 ml saline) by i.p injection. Sixteen weeks after these treatments the mice were killed and pulmonary adenomas were counted The statistical significance of bioassay data was determined by student's test.

TABLE 9
Effect of Drug on NNK-induced lung tumorigenesis

| Group | Tumor incidence (%) |
|---|---|
| No drug | 100 |
| HHT | 35.8 ± 4.5 |
| HHT + IND | 20.6 ± 3.8* |
| HHT + YLE | 30.6 ± 4.8 |
| HHT + LIK | 31.8 ± 4.9 |
| HHT + MAT | 28.5 ± 3.9* |
| HHT + APN | 32.8 ± 4.2 |
| HHT + PYE | 29.2 ± 4.2* |

*P < 0.05 compared with HHT group

Data of Table 9 indicated that HHT has a significant inhibitory effect against lung tumor and could decrease tumor incidence. Therefore, HHT could prevent cancer Other drugs could increase the effect of HHT.

### Example 17.

Effect of Drug on Peroxidation

The peroxidized lipids were deleterious to health as they induce cross-linking of DNA and proteins damage The oxidized low density lipoproteins were efficiently phagocytosed by macrophages through scavenger receptors.

Methods

The liver microsome (1 mg protein) in 0.15 M KCl, 0 25 M Tris-HCl buffer, pH 7.5, 2 mμ ADP, 10 μM FeSO₄ and Drug B (50 μM) were incubated at 37° C. for 5 min The reactions were terminated after 30 min by added 2 ml thiobarbituric acid (TBA) and content of the malondialdehyde (MDA) formed was determined.

TABLE 10
Effect of Drug on lipid peroxidation

| Group | Nmoles MDA/mg protein |
|---|---|
| No drug | 25.4 ± 0.3 |
| HHT | 8.5 ± 1.0 |
| HHT + IND | 4.0 ± 0.6* |
| HHT + YLE | 8.0 ± 1.1 |
| HHT + LIK | 7.5 ± 1.9 |
| HHT + MAT | 6.5 ± 0.9* |
| HHT + APN | 7.0 ± 1.6 |
| HHT + PYE | 6.0 ± 1.1* |

*P < 0.01 compared with HHT group
Results are mean ± SD value from 20 rats

Data of table 10 indicated that HHT could significantly inhibit lipid peroxidation. Decreasing lipid peroxidation could prevent cancer. Other drugs could increase the effect of HHT.

### EXAMPLE 18.

Effect of Drug on Inhibition of Carcinogen-DNA Binding

Inhibition of carcinogen-DNA binding was one of biochemical markers for screening potential chemopreventive agents by National Cancer Institute of the US

Methods

Cells were maintained in 75-cm² tissue culture flask with media supplemented with retinoic acid (0 1 μg/ml) and epinephrine (0 5 μg/ml) The cells were passage when they reached approximately 80% confluence and were maintained at 37° C. in 5% CO₂. Cells were plated at a density of 5×10⁵ cells/well in 6-well tissue culture plates and allowed to incubated for 18 h at 37° C. in 5% CO₂ Following incubation, the DNA was harvested and resuspended in Tris-EDTA buffer RNA was removed by treating with RNase T1 and RNase A at 37° C. for 1 h. An aliquot was used for determining the DNA content by the absorbency at 260 nm, and the rest of the sample was used to determine the radioactivity; the percentage inhibition of carcinogen-DNA binding was determined by measuring the cpm/mg of DNA in the carcinogen

TABLE 11
Effect of Drug on inhibition of carcinogen-DNA binding

| Group | Inhibition (%) |
|---|---|
| No drug | — |
| HHT | 56.8 ± 6.2 |
| HHT + IND | 40.8 ± 8.3* |
| HHT + YLE | 50.8 ± 9.2 |
| HHT + LIK | 48.8 ± 6.8* |
| HHT + MAT | 42.8 ± 9.6* |
| HHT + APN | 51.8 ± 10.2 |
| HHT + PYE | 54.8 ± 7.6 |

*P < 0.01 compared with HHT group

Data of table 11 indicated that HHT significantly inhibits carcinogen-DNA binding and other drugs could increase the effect of HHT

### Example 19.

Safety of HHT

(A) LD₅₀ The LD₅₀ of HHT in mice (I P ) was found to be 3 17±0.19 mg/kg. Toxic doses for dogs 0 16 mg/kg/day×7 of HHT was established as the toxic doses. Toxic doses for mice In 38 normal mice after injection of HHT of 2 mg/kg/day×5 with the observation period of 5 days about 50% of the mice died.

LD₅₀ of HHT is much higher than majority anticancer drugs The toxicology data of HHT mean that HHT is safe drug for treatment of cancer cells.

(B) Dose. HHT was administered at a dose of 1˜5 mg/m² as infusion through venous or injection MAT, APN, YLE, LIK, PUN and IND were administered at a dose of 25˜250 mg orally

The preparation of drugs, which can be accomplished by the extraction methods set forth above or any conventional methods for extracting the active principles from the plants. The novelty of the present invention resides in the mixture of the active principles in the specified proportions to produce drugs, and in the preparation of dosage units in pharmaceutically acceptable dosage form The term “pharmaceutically acceptable dosage form” as used hereinabove includes any suitable vehicle for the administration of medications known in the pharmaceutical art, including, by way of example, capsules, tablets, syrups, elixirs, and solutions for parenteral Injection with specified ranges of drugs concentration.

In addition, the present invention provides novel methods for treatment of cancer cells with produced safe pharmaceutical agent

It will thus be shown that there are provided compositions and methods which achieve the various objects of the invention and which are well adapted to meet the conditions of practical use

As various possible embodiments might be made of the above invention, and as various changes might be made in the embodiments set forth above, it is to be understood that all matters herein described are to be interpreted as illustrative and not in a limiting sense.

What is claimed as new and desired to be protected by Letter Patent is set forth in the appended claim:

1. A safe natural drug comprises Homoharringtonine (HHT) or HHT combined with other ingredients, which include Matrine (MAT), Apigenin (APN), Yejuhua lactone (YLE), Lipopolysaccharide of Kelp (LIK), Puerarin (PUN) and Indirubin (IND), for diverting human cancer cells to resemble normal cells, inducing apoptosis of cancer cells and inhibiting cancer growth

2. A safe natural drug, according to claim 1, wherein said Homoharringtonine is extracted from cells, tissue cultures or natural sources of Cephalotaxus sinensis Li or Cephalotaxus hainanensis Li or Cephalotaxus fortune Hook, or other Cephalotaxus species.

3. A process for a safe natural drug in accordance with claim 2 wherein said cell or tissue culture comprising:
(a) Parts of stems, leaves, skins or roots of Cephalotaxus species are surface disinfected by treated in 70% ethanol for 10 minutes and followed by 0.1 HgCl₂ for 3 minutes;
(b) plant materials are washed five times for 10 minutes each by sterilized water;
(c) parts of plant are cut into small pieces (0.5˜1 mm) and put pieces to Murashige and Skoog's (MS) medium and supplemented with derivative of gibberellin, naphthalene-acid (NAA), phenylolanine, tyrosine, kinetin and sucrose;
(d) pH of medium is adjusted to 5 7˜5 8;
(e) agar is added to medium,
(f) callus tissues are collected from agar media and suspension cultured cells are harvested by filtration and cultured in MS medium;
(g) cultures are kept in culture room at 26° C.;
(h) friable callus tissues are obtained,
(i) callus tissues are inoculated into MS medium containing derivative of gibberllin, NAA, kinetin and surcrose,
(j) callus tissues are subcultured at 26° C. for 35 days on rotary shaker operated at 80 rpm;
(k) cells are subcultured into fresh medium of same composition every 2 weeks and maintained at 120 rpm at 26° C.,
(l) packed cell volume (PCV) fresh weight (FW), dry weight (DW), concentration of HHT and concentration of sugar are determined every 5^{th} day,
(m) cells are harvested and dried

4. A safe natural drug of claim 1, wherein the amount sufficient to induce diversion of cancer cells to resemble normal cells, is about 1-5 mg/m² by infusion through venous or injection of HHT

5. A safe natural drug of claim 1, wherein the amount sufficient to induce apoptosis of cancer cells, is about 1-5 mg/m² by infusion through venous or injection of HHT

6. A safe natural drug of claim 1, wherein the amount sufficient to inhibit cancer cells proliferation, is about 1-5 mg/m² by infusion through venous or injection of HHT.

7. A safe natural drug of claim 1, wherein the amount sufficient to inhibit transplanted tumor is about 1-5 mg/m² by infusion through venous or injection of HHT.

8. A safe natural drug of claim 1, wherein the amount sufficient to decrease tyrosine kinase is about 1-5 mg/m² by infusion through venous or injection of HHT.

9. A safe natural drug of claim 1, wherein the amount sufficient to inhibit tumor incidence in vivo is about 1-5 mg/m² by infusion through venous or injection of HHT.

10. A safe natural drug of claim 1, wherein the amount sufficient to inhibit carcinogen-DNA binding is about 1-5 mg/m² by infusion through venous or injection of HHT.

11. A safe natural drug of claim 1, wherein the amount sufficient to inhibit peroxidation is about 1-5 mg/m² by infusion through venous or injection of HHT.

12. A safe natural drug of claim 1, wherein the amount sufficient to induce diversion of cancer cells to resemble normal cells, is 1-5 mg/m² by infusion through venous or injection of HHT and added about 25˜250 mg of MAT orally.

13. A safe natural drug of claim 1, wherein the amount sufficient to induce diversion of cancer cells to resemble normal cells, is 1-5 mg/m² by infusion through venous or injection of HHT and about 25˜250 mg of APN orally.

14. A safe natural drug of claim 1, wherein the amount sufficient to induce diversion of cancer cells to resemble normal cells, is 1-5 mg/m² by infusion through venous or injection of HHT and added about 25˜250 mg of YLE orally

15. A safe natural drug of claim 1, wherein the amount sufficient to induce diversion of cancer cells to resemble normal cells, is 1-5 mg/m² by infusion through venous or injection of HHT and added about 25˜250 mg of LIK orally

16. A safe natural drug of claim 1, wherein the amount sufficient to induce diversion of cancel cells to resemble normal cells, is 1-5 mg/m² by infusion through venous or injection of HHT and added about 25˜250 mg of PUN orally

17. A safe natural drug of claim 1, wherein the amount sufficient to induce diversion of cancer cells to resemble normal cells, is 1-5 mg/m² by infusion through venous or injection of HHT and added about 25˜250 mg of IND orally

18. A safe natural drug of claim 1, wherein the amount sufficient to induce apoptosis of cancer cells, is 1-5 mg/m2 by infusion through venous or injection of HHT and added about 25˜250 mg of MAT orally

19. A safe natural drug of claim 1, wherein the amount sufficient to induce apoptosis of cancer cells, is 1-5 mg/m² by infusion through venous or injection of HHT and added about 25˜250 mg of APN orally

20. A safe natural drug of claim 1, wherein the amount sufficient to induce apoptosis of cancer cells, is 1-5 mg/m² by infusion through venous or injection of HHT and added about 25˜250 mg of YLE orally

21. A safe natural drug of claim 1, wherein the amount sufficient to induce apoptosis of cancer cells, is 1-5 mg/m² by infusion through venous or injection of HHT and added about 25˜250 mg of LIK orally

22. A safe natural drug of claim 1, wherein the amount sufficient to induce apoptosis of cancer cells, is 1-5 mg/m² by infusion through venous or injection of HHT and added about 25˜250 mg of PUN orally

23. A safe natural drug of claim 1, wherein the amount sufficient to induce apoptosis of cancer cells, is 1-5 mg/m² by infusion through venous or injection of HHT and added about 25˜250 mg of IND orally

24. A safe natural drug of claim 1, wherein the amount sufficient to inhibit cancer cells proliferation, is 1-5 mg/m² by infusion through venous or injection of HHT and added about 25˜250 mg of MAT orally

25 A safe natural drug of claim 1, wherein the amount sufficient to inhibit cancer cells proliferation, is 1-5 mg/m² by infusion through venous or injection of HHT and added about 25˜250 mg of APN orally

26. A safe natural drug of claim 1, wherein the amount sufficient to inhibit cancer cells proliferation, is 1-5 mg/m² by infusion through venous or injection of HHT and added about 25˜250 mg of YLE orally

27. A safe natural drug of claim 1, wherein the amount sufficient to inhibit cancer cells proliferation, is 1-5 mg/m² by infusion through venous or injection of HHT and added about 25˜250 mg of LIK orally

28 A safe natural drug of claim 1, wherein the amount sufficient to inhibit cancer cells proliferation, is 1-5 mg/m² by infusion through venous or injection of HHT and added about 25˜250 mg of PUN orally

29. A safe natural drug of claim 1, wherein the amount sufficient to inhibit cancer cells proliferation, is 1-5 mg/m² by infusion through venous or injection of HHT and added about 25˜250 mg of IND orally

30. A safe natural drug of claim 1, wherein the amount sufficient to inhibit transplanted tumor is 1-5 mg/m² by infusion through venous or injection of HHT and added about 25-250 mg of MAT orally

31. A safe natural drug of claim 1, wherein the amount sufficient to inhibit transplanted tumor is 1-5 mg/m² by infusion through venous or injection of HHT and added about 25-250 mg of APN orally

32 A safe natural drug of claim 1, wherein the amount sufficient to inhibit transplanted tumor is 1-5 mg/m² by infusion through venous or injection of HHT and added about 25-250 mg of YLE orally

33. A safe natural drug of claim 1, wherein the amount sufficient to inhibit transplanted tumor is 1-5 mg/m² by infusion through venous or injection of HHT and added about 25-250 mg of LIK orally

34. A safe natural drug of claim 1, wherein the amount sufficient to inhibit transplanted tumor is 1-5 mg/m² by infusion through venous or injection of HHT and added about 25-250 mg of PUN orally.

35. A safe natural drug of claim 1, wherein the amount sufficient to inhibit transplanted tumor is 1-5 mg/m² by infusion through venous or injection of HHT and added about 25-250 mg of IND orally

36. A safe natural drug of claim 1, wherein the amount sufficient to decrease tyrosine kinase is about 1-5 mg/m² by infusion through venous or injection of HHT and added about 25˜250 mg of MAT orally

37. A safe natural drug of claim 1, wherein the amount sufficient to decrease tyrosine kinase is about 1-5 mg/m² by infusion through venous or injection of HHT and added about 25˜250 mg of APN orally

38. A safe natural drug of claim 1, wherein the amount sufficient to decrease tyrosine kinase is about 1-5 mg/m² by infusion through venous or injection of HHT and added about 25˜250 mg of YLE orally

39. A safe natural drug of claim 1, wherein the amount sufficient to decrease tyrosine kinase is about 1-5 mg/m² by infusion through venous or injection of HHT and added about 25˜250 mg of LIK orally

40. A safe natural drug of claim 1, wherein the amount sufficient to decrease tyrosine kinase is about 1-5 mg/m² by infusion through venous or injection of HHT and added about 25˜250 mg of PUN orally

41. A safe natural drug of claim 1, wherein the amount sufficient to decrease tyrosine kinase is about 1-5 mg/m² by infusion through venous or injection of HHT and added about 25˜250 mg of IND orally

42. A safe natural drug of claim 1, wherein the amount sufficient to inhibit tumor incidence in vivo is about 1-5 mg/m² by infusion through venous or injection of HHT and added about 25˜250 mg of MAT orally

43 A safe natural drug of claim 1, wherein the amount sufficient to inhibit tumor incidence in vivo is about 1-5 mg/m² by infusion through venous or injection of HHT and added about 25˜250 mg of APN orally

44. A safe natural drug of claim 1, wherein the amount sufficient to inhibit tumor incidence in vivo is about 1-5 mg/m² by infusion through venous or injection of HHT and added about 25˜250 mg of YLE orally.

45 A safe natural drug of claim 1, wherein the amount sufficient to inhibit tumor incidence in vivo is about 1-5 mg/m² by infusion through venous or injection of HHT and added about 25˜250 mg of LIN orally

46 A safe natural drug of claim 1, wherein the amount sufficient to inhibit tumor incidence in vivo is about 1-5 mg/m² by infusion through venous or injection of HHT and added about 25˜250 mg of PUN orally

47. A safe natural drug of claim 1, wherein the amount sufficient to inhibit tumor incidence in vivo is about 1-5 mg/m² by infusion through venous or injection of HHT and added about 25˜250 mg of IND orally

48. A safe natural drug of claim 1, wherein the amount sufficient to inhibit carcinogen-DNA binding is about 1-5 mg/m² by infusion through venous or injection of HHT and added about 25˜250 mg of MAT orally.

49. A safe natural drug of claim 1, wherein the amount sufficient to inhibit carcinogen-DNA binding is about 1-5 mg/m² by infusion through venous or injection of HHT and added about 25˜250 mg of APN orally

50 A safe natural drug of claim 1, wherein the amount sufficient to inhibit carcinogen-DNA binding is about 1-5 mg/m² by infusion through venous or injection of HHT and added about 25˜250 mg of YLE orally.

51. A safe natural drug of claim 1, wherein the amount sufficient to inhibit carcinogen-DNA binding is about 1-5 mg/m² by infusion through venous or injection of HHT and added about 25˜250 mg of LIN orally.

52 A safe natural drug of claim 1, wherein the amount sufficient to inhibit carcinogen-DNA binding is about 1-5 mg/m² by infusion through venous or injection of HHT and added about 25˜250 mg of PUN orally

53. A safe natural drug of claim 1, wherein the amount sufficient to inhibit carcinogen-DNA binding is about 1-5 mg/m² by infusion through venous or injection of HHT and added about 25˜250 mg of IND orally

54. A safe natural drug of claim 1, wherein the amount sufficient to inhibit peroxidation is about 1-5 mg/m² by infusion through venous or injection of HHT and added about 25˜250 mg of MAT orally

55. A safe natural drug of claim 1, wherein the amount sufficient to inhibit peroxidation is about 1-5 mg/m² by infusion through venous or injection of HHT and added about 25˜250 mg of APN orally

56 A safe natural drug of claim 1, wherein the amount sufficient to inhibit peroxidation is about 1-5 mg/m² by infusion through venous or injection of HHT and added about 25˜250 mg of YLE orally.

57 A safe natural drug of claim 1, wherein the amount sufficient to inhibit peroxidation is about 1-5 mg/m² by infusion through venous or injection of HHT and added about 25˜250 mg of LIK orally.

58. A safe natural drug of claim 1, wherein the amount sufficient to inhibit peroxidation is about 1-5 mg/m² by infusion through venous or injection of HHT and added about 25˜250 mg of PUN orally

59 A safe natural drug of claim 1, wherein the amount sufficient to inhibit peroxidation is about 1-5 mg/m² by infusion through venous or injection of HHT and added about 25˜250 mg of IND orally

60 A process for producing natural pharmaceutical composition in accordance with claim 1 wherein said producing Matrine (MAT) comprising:
(a) extracting the dried powder of root of Sophora flavescens Alt with methanol;
(b) filtering the extract,
(c) concentrating the filtrate and residue was obtained,
(d) residue was dissolved in HCI and adjusted pH to 3 5;
(e) NaOH was added to HCI solution and adjusted pH to 13;
(f) solution of pH 13 was extracted by CH₂Cl₂,
(g) CH₂Cl₂ was recovered under reduced pressure and residue was dissolved in CHCl₃;
(h) adding diethyl ether to CHCl₃ and then mixture was filtered,
(i) filtrate was concentrated and syrup was obtained;
(j) the syrup was chromatographic column packed with alumina again;
(k) column as eluted with oil ether-acetone,
(l) elution was concentrated and residue obtained,
(m) acetone was added to residue and crystallized;
(o) crystals were recrystallized in acetone; and
(p) crystals were dried under vacuum;

61 A process for producing natural pharmaceutical composition in accordance with claim 1 wherein said producing Apiin (APN) comprising
(a) extracting the dried powder of Apium graveolens L with cold ethanol,
(b) extract was filtrated,
(c) filtrate (1) was concentrated under reduced pressure and filtrated,
(c) filtrate (2) was cooled and precipitate was obtained,
(e) precipitate was washed by diethyl ether and acetone;
(f) precipitate was dissolved in hot water;
(g) Pb (OAC)₂ was added to hot water and precipitate was formed;
(h) the mixture was filtered;
(i) NaOH was added to filtrate and precipitate was formed,
(j) precipitate was washed with water and suspended on EtOH;
(k) H₂S was gassed to suspension of EtOH and filtered;
(l) filtrate was concentrated under reduced pressure and crystals were formed;
(m) crystals were refined by recrystallization in cold ethanol; and
(n) crystals were dried under vacuum

62 A process for producing natural pharmaceutical composition in accordance with claim 1 wherein said producing Indirubin (IND) comprising:
(a) dried powder of Baphicanthus cusia (Ness) Bremek, or Isatis tinctoria L, or Isatis indigotica Fort, or Polygonum tinctorium Ait was extracted with hot water,
(b) extract was filtered and filtercake extracted with methanol,
(c) methanol was recovered under reduced pressure and residue obtained,
(d) the residue was extracted with chloroform,
(e) the chloroform was recovered and residue was chromatographed on silica gel;
(f) silica gel eluted by chloroform,
(g) chloroform was concentrated and crystals were obtained,
(h) crystals were refined by recrystallization in chloroform, and
(i) crystals were dried under vacuum.

63. A process as claimed in claim 1 wherein the extracrting Yejuhua-flavonoid (YLE) comprising:
(a) extracting a ground of Chrysatnhemum indicum L with 95% ethanol at room temperature for 24 hours,
(b) filtering the above mixture and separating a filtrate A from a filtercake;
(c) percolating the filtercake with ethanol and collecting a filtrate B;
(d) combining filtrates A and B and distilling them under reduced pressure to recover ethanol and an aqueous residue,
(e) acetic ether was added to residue and refluxed bath for 6 hours,
(f) acetic ether was recovered under reduced pressure and crystals were formed;
(g) crystals were recrystallized in acetic ether,
(h) crystals were dried under vacuum, and
(i) the product is YLE

64. A process as claimed in claim 1 wherein said the extracting Lipopolysaccharide of Kelp (LIK) comprising:
(a) extracting a dried and ground of kelp with water;
(b) water extraction was repeated one and the two extracts were combined,
(c) extracts was filtered and filtrate was concentrated under reduced pressure;
(c) 95% ethanol was added to concentrate and precipitate was formed;
(d) mixture was filtered and precipitate was washed with ethanol, acetone and ether consecutively;
(f) powder was dried under vacuum; and
(g) the product is Lipopolysaccharides of Kelp

65. A process as claimed in claim 1 wherein the extracting Puerain (PUN) comprising,
(a) extracting a dried and ground of Pueraria lobata (wild.) Ohwi or Pueraria thomsanii Benth with ethanol,
(b) extraction was repeated twice with ethanol and extract was combined,
(c) ethanol was recovered under reduced pressure and residue was dried;
(d) residue was chromatographed on alumina and eluted with water-saturated butanol;
(e) water-saturated butanol as developing solvent to separated into color bonds which determined by U V,
(g) special band eluted with butanol-pyridine;
(h) butanol-pyridine was concentrated under reduced pressure and crystals were obtained;
(i) crystals were recrystallized from ethanol, and
(j) the product is Pureatin

66. A method of extracting homoharringtonine (HHT) from cultured plant tissue and natural plant material that contains HHT, comprising the steps of:
Contacting the plant material for a selected period of time with a solvent whereby at least some of said HHT is soluble and transported into said solvent thereby forming a crude extract;
Adjusting said pH of crude extract for specifically separating said crude HHT,
Desorbing said crude HHT sequentially from said adsorbent from said adsorbent by flowing a series of eluant mixtures making up a step gradient elution over said column and collecting each of individual eluant mixtures of said step gradient elution flowing through said column wherein each of said individual eluant mixture contains pure HHT compound.

67. The method of claim 66, wherein said solvent s are ethanol, methyl alcohol, water, HCI and chloroform

68. The method of claim 66, wherein said eluant is chloroform and methanol.

69. The method of claim 66, wherein said adsorbent is alumina

70. The method of claim 66, wherein said extracting HHT comprising the steps of
(a) extracting a ground cultured plant tissue or cells or plant selected from the group consisting of Cephalotaxus fortunei Hook, C. sinensis Li, C hainanensis and C wilsoniana or other Cephalotaxus species with 90% ethanol at room temperature for 24 hours,
(b) filtering the above mixture and separating a filtrate A from a filtercake;
(c) percolating the filtercake with ethanol and collecting a filtrate B;
(d) combining filtrates A and B and distilling them under reduced pressure to recover ethanol and an aqueous residue;
(e) adjusting the pH of the residue to 2 5,
(f) separating solids from the resulting mixture by filtration to yield a filtrate;
(g) adjusting the pH of the filtrate of step (f) to 9 5;
(h) extracting the alkaline solution of step (g) five times with chloroform, combining all the chloroform extracts and distilling them to recover alkaloids,
(i) dissolving the alkaloids in citric acid, dividing the solution into three portions, and adjusting the pH of the three portions to 7, 8, and 9,
(j) extracting the portions of pH 8 and 9 with chloroform;
(k) distilling the chloroform extract to yield raw harringtonine;
(l) purifying said harringtonine by crystallizing the same in pure ethanol and recrystallizing the same in diethyl ether,
(m) combining the portion of pH 7 of step (i) and the mother liquors resulting from step (l),
(n) passing the solution of step (m) through a chromatographic column packed with alumina, flushing said column with chloroform and subsequently with a chloroform-methanol mixture to yield a mixture of harringtonine and homoharringtonine, and
(o) separating the homoharringtonine from harringtonine by countercurrent distribution with chloroform and pH 5 buffer The methyl alcohol added to first fraction,
(q) the mixture was concentrated under reduced pressure and crystallization is obtained;
(r) the crystallization was purified by recrystallization in methyl alcohol; and
(s) the crystal was dried in vacuum

71 A process for producing KHT by semi-synthesis comprising:
(a) extracting a ground cultured plant tissue or plant selected from the group consisting of Cephalotaxus fortunei Hook, C sinensis Li, C hainanensis, C wilsoniana and other Cephalotaxus species with 90% ethanol at room temperature for 24 hours;
(b) the ethanol was recovered under reduced pressure;
(c) tartaric acid was added to concentrated ethanol solution,
(d) ammonia water was added to acidic solution and adjusted pH to 9;
(e) pH 9 solution was filtered and yielded filtrate,
(f) filtrate was extracted with CHCl₃;
(g) CHCl₃ was recovered and residue was obtained;
(h) residue was chromatographed packed with alumna and eluted by CHCl₃-MeOH;
(i) elute was concentrated under reduced pressure and residue was dried under vacuum; and
(j) the dried residue is Cephalotaxus (CEP), which used for semi-synthesis of HHT.

72. The method of claim 71, wherein said semi-synthesis of HHT comprising:
(a) benzene-α-acetone-Na was put into benzene;
(b) mixture was stirred then was dissolved in pyridine at stirred at 0° C.;
(c) oxalic chloride was added to solution of pyridine,
(d) solution warmed to room temperature and stand overnight;
(e) the solution was added to CH₂Cl₂ and cooled to 0° C.;
(f) CEP and pyridine were added to cold CH₂Cl₂ solution,
(g) Mixture (2) was washed with 10% Na₂CO₃ and saturated NaCl solution;
(h) Solvents were evaporated and solid α-ketoester-harringtonine obtained;
(i) CH3CHBrCOOEt and activated zin dust were added to α-ketoester-harringtonine and mixture (3) was obtained,
(j) CHCH₃ and H₂O and solid Na₂CO₃ were added to the mixture (3),
(k) CHCl₃ was evaporated under reduced pressure and residue was obtained;
(l) the residue was chromatography picked with alumina;
(m) column eluted with chloroform and followed by chloroform-methanol;
(n) solvents were recovered under reduced pressure and solid was obtained;
(o) solid was dissolved in ethanol,
(p) ethanol was recovered under reduced pressure and crystals were obtained;
(q) crystals were recrystallized in diethyl ether;
(r) crystals were dried under vacuum, and
(s) the product is HHT.
